(19) United States
(12) Patent Application Publication
(10) Pub. No.: US 2015/0043003 A1
(43) Pub. Date: Feb. 12, 2015

(54) OPTICAL COHERENCE TOMOGRAPHY AND CONTROL METHOD FOR THE SAME

(71) Applicants: Jungho Chung, Seoul (KR); Sungho Hong, Seoul (KR)

(72) Inventors: Jungho Chung, Seoul (KR); Sungho Hong, Seoul (KR)

(73) Assignee: LG ELECTRONICS INC., Seoul (KR)

(21) Appl. No.: 14/383,464

(22) PCT Filed: Apr. 18, 2012
(86) PCT No.: PCT/KR2012/002934

§ 371 Date: Sep. 5, 2014

(51) Int. Cl. G01B 9/02; A61B 5/00

(52) U.S. Cl. CPC G01B 9/02091; G01B 9/02041; A61B 5/0066
USPC 356/479

(57) ABSTRACT

Disclosed is an optical coherence tomography which includes: a light source unit for outputting light; a light splitting unit for splitting the light, which is reflected from a sample, into visible light and OCT source beam; a detection unit for detecting the visible light and the OCT source beam; and a display unit for displaying a first image based on the detected visible light and a second image based on the detected OCT source beam.

### TECHNICAL FIELD

The present invention relates to an optical coherence tomography (OCT) and a control method for the same.

### BACKGROUND ART

Exemplary medical apparatuses, which can acquire images of inner parts of a human body, include X-ray imaging apparatuses, magnetic resonance imaging (MRI) apparatuses, computer tomographic (CT) apparatuses, ultrasound imaging apparatuses, etc.

The X-ray imaging apparatus has a disadvantage in that it uses radiation, which has a detrimental effect on a human body.

The MRI apparatus and the CT apparatus have a disadvantage in that they are large-scaled and expensive, such that they are not commonly used but limitedly used in some large hospitals.

In addition, the MRI apparatus has another disadvantage in that it is difficult to use for patients having an medical material or structure made of an iron, either inside or outside their body.

The ultrasound imaging apparatus, which is cheaper than the MRI apparatus and the CT apparatus, has a disadvantage of low resolution.

Nowadays, an optical coherence tomography (OCT) has been developed, that has a simpler structure than the CT apparatus or the MRI apparatus and that can provide higher resolution than the ultrasound imaging apparatus.

The OCT, which is also called an optical imaging apparatus, is a real-time imaging system of high resolution, which can image the section of a microstructure inside a living epidermal tissue. In other words, the OCT uses a medical imaging technique of imaging the inside of a living body in a non-contact manner, based on an optical coherence principle of near-infrared wavelengths of white light. Recently, researches have been actively made on it.

FIG. 1

FIG. 1 is a view showing an example of an OCT.

Referring to FIG. 1, the OCT can obtain an OCT scanning image by inputting light rays, which are reflected from a sample, directly to an OCT system.

The OCT system can acquire a tomographic image of the sample, based on the input light rays. The OCT scanning image may include a tomographic image of the sample.

However, in the above method, the scanning point of an OCT probe can only be determined according to inaccurate information based on the estimated position of the OCT probe. That is, it is difficult for the user to check an accurate position of the OCT probe with his/her naked eyes. As a result, this method does not allow the user to determine whether an exact desired point has been scanned.

### DISCLOSURE OF INVENTION

### Technical Problem

Therefore, the present disclosure has been made to solve the aforementioned problems in the prior art.

Specifically, an object of the present disclosure is to provide a method of acquiring a visible image of a scanning point of an OCT probe.

Another object of the present disclosure is to provide a method of allowing a user to easily check a scanning point of an OCT probe with his/her naked eyes through a visible image and to easily obtain an OCT scanning image of a desired point.

### Solution to Problem

According to an aspect of the present invention, there is provided an optical coherence tomography (OCT), which may include: a light source unit for outputting light; a light splitting unit for splitting the light, which is reflected from a sample, into visible light and OCT source beam; a detection unit for detecting the visible light and the OCT source beam; and a display unit for displaying a first image based on the detected visible light and a second image based on the detected OCT source beam.

According to another aspect of the present invention, the light splitting unit may include a dichroic mirror.

According to a further aspect of the present invention, the dichroic mirror may transmit the visible light and reflect the OCT source beam or may transmit the OCT source beam and reflect the visible light.

According to a still further aspect of the present invention, the light splitting unit may include a panel, one surface of which transmitting the visible light and reflecting the OCT source beam, the other surface of which reflecting the visible light.

According to a still further aspect of the present invention, the light splitting unit may include a panel, one surface of which transmitting the OCT source beam and reflecting the visible light, the other surface of which reflecting the OCT source beam.

According to a still further aspect of the present invention, the detection unit may include a visible light camera for photographing an image based on the visible light.

According to a still further aspect of the present invention, the visible light camera may include at least one of charge coupled device (CCD) cameras and complementary metal-oxide semiconductor (CMOS) cameras.

According to a still further aspect of the present invention, the detection unit may include an OCT system for acquiring an image based on the OCT source beam.

According to a still further aspect of the present invention, the display unit may display the first image and the second image in the overlapping manner.

According to a still further aspect of the present invention, the first image may include a visible image of the sample, while the second image may include a tomographic image of the sample.

According to a still further aspect of the present invention, the OCT may further include a user input unit for receiving an input of selecting some region of the first image, wherein the display unit may display the second image corresponding to the selected region.

According to an aspect of the present invention, there is provided a control method for an optical coherence tomography (OCT), which may include: radiating light to a sample; splitting the light, which is reflected from the sample, into visible light and OCT source beam; displaying a visible image of the sample based on the visible light; and displaying a tomographic image of the sample based on the OCT source beam.

According to another aspect of the present invention, the splitting of light may includes transmitting the visible light and reflecting the OCT source beam at a dichroic mirror.

According to a further aspect of the present invention, the splitting of light may includes reflecting the visible light and transmitting the OCT source beam at a dichroic mirror.

### Advantageous Effects of Invention

The present disclosure can solve the foregoing problems in the prior art.

Specifically, according to the present disclosure, it is possible for the user to acquire a visible image of a scanning point of an OCT probe.

Moreover, according to the present disclosure, it is possible for the user to easily check a scanning point of an OCT probe with his/her naked eyes through a visible image and to easily obtain an OCT scanning image of a desired point.

### BRIEF DESCRIPTION OF DRAWINGS

FIG. 1 is a view showing an example of an OCT.

FIG. 2 is a block diagram showing an OCT according to an embodiment of the present disclosure.

FIG. 3 is a flowchart showing an example of a method of the OCT displaying an image of a sample.

FIG. 4 is a view showing an example of a method of the OCT splitting light reflected from the sample.

FIG. 5 is a view showing another example of the method of the OCT splitting light reflected from the sample.

FIG. 6 is a view showing a further example of the method of the OCT splitting light reflected from the sample.

FIG. 7 is a view showing a still further example of the method of the OCT splitting light reflected from the sample.

FIGS. 8a to 8c are views showing an example of a method of the OCT displaying a first image and a second image.

FIGS. 9a and 9b are views showing another example of the method of the OCT displaying the first image and the second image.

FIGS. 10a and 10b are views showing a further example of the method of the OCT displaying the first image.

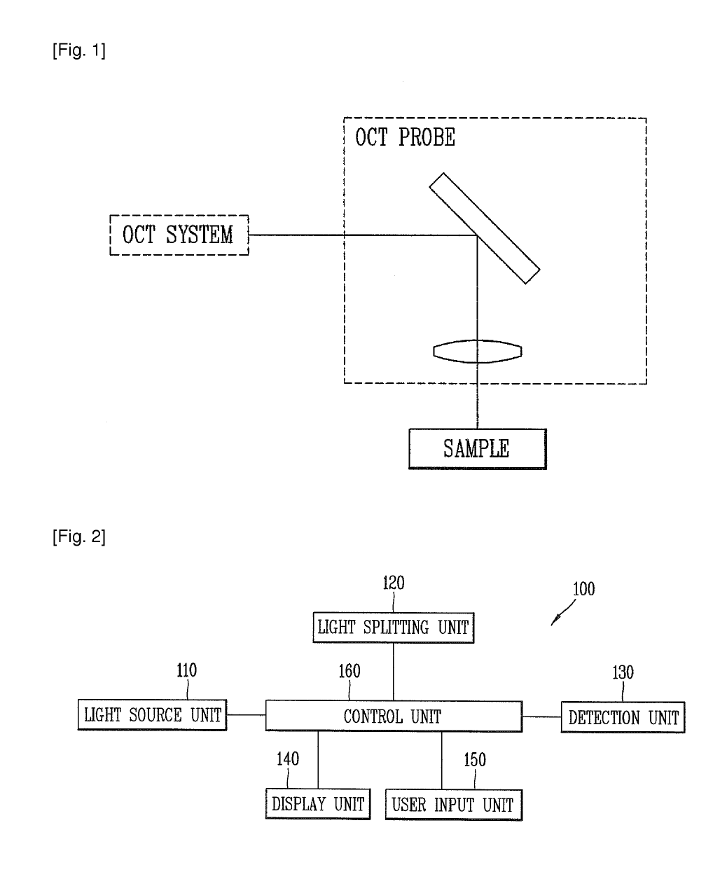

### MODE FOR THE INVENTION

Technical and scientific terms used herein are for the purpose of describing particular embodiments only and are not intended to be limiting the present invention. Unless defined otherwise, all the technical and scientific terms used herein should be construed as the same meanings as commonly understood by those skilled in the art and should not be construed as excessively inclusive meanings or excessively exclusive meanings. If technical and scientific terms used herein do not expressly represent the ideas of the present invention, they should be translated as the proper ones so that those skilled in the art would understand such terms. General terms used herein should be construed as their lexical meanings or understood in the context and should not be construed as excessively exclusive meanings.

As used herein, the singular forms are intended to include the plural forms as well, unless the context clearly indicates otherwise. It will be further understood that the terms “comprises”, “comprising”, “includes” and “including”, when used herein, specify the presence of stated elements or steps, but do not preclude the absence of some elements or steps thereof or the presence or addition of other elements or steps thereof.

As used herein, “modules”, “units” and “portions” which describe elements are for the purpose of the ease of description, and thus are not intended to distinguish one element from another.

As used herein, although the terms “first”, “second”, etc. may be used herein to describe various elements, these elements should not be limited by these terms. These terms are only used to distinguish one element from another. For example, a first element could be termed a second element, and, similarly, a second element could be termed a first element, without departing from the scope of the present invention.

Reference will now be made in detail to the preferred embodiments of the present invention, examples of which are illustrated in the accompanying drawings. Throughout the drawings, same or like elements are given same or like reference numerals, and a duplicate description thereof will be omitted.

In some instances, well-known process steps and/or structures are not described in detail since such description would detract from the clarity and concision of the disclosure of the invention. It is to be noted that the ideas of the present invention are better understood by the accompanying drawings but are not limited thereto.

FIG. 2

FIG. 2 is a block diagram showing an OCT 100 according to an embodiment of the present disclosure.

The OCT 100 may include a light source unit 110, a light splitting unit 120, a detection unit 130, a display unit 140, a user input unit 150, and a control unit 160. As the elements shown in FIG. 2 are not essential, an OCT which has more elements or less elements can be implemented.

The elements will now be described in sequence.

The light source unit 110 can radiate light to a sample. The light source unit 110 can output light of a wide band. The light source unit 110 can also output light having a small coherence length, such as about a few tens μm. In addition, the light emitted from the light source unit 110 may have a wavelength band which has a low absorption factor with respect to a material in the sample and which can be deeply penetrating.

The light splitting unit 120 can split the light, which is radiated to the sample by the light source unit and reflected from the sample. For example, the light splitting unit 120 can split the reflected light into visible light and OCT source beam. The OCT source beam is the light which contains information required to acquire an OCT scanning image.

In the meantime, the light splitting unit 120 can be implemented by a dichroic mirror, prism, etc.

The detection unit 130 can acquire an image based on the light split by the light splitting unit. For example, the detection unit 130 can acquire a first image based on the visible light. Here, the detection unit 130 may use a visible light camera in order to acquire the first image based on the visible light. The visible light camera can photograph an image based on the visible light. Moreover, exemplary visible light cameras may include charge coupled device (CCD) cameras, complementary metal-oxide semiconductor (CMOS) cameras, etc.

Meanwhile, the camera can acquire an image or a moving image.

In addition, the detection unit 130 can acquire a second image based on the OCT source beam. The second image may include a tomographic image of the sample. Here, the detection unit 130 may use an OCT system in order to acquire the second image based on the OCT source beam. The OCT system indicates a configuration which enables the acquisition of the second image based on the OCT source beam.

The display unit 140 can display the first image, the second image, etc., which are acquired by the detection unit 130. For example, the display unit 140 can display a visible image of the sample, a tomographic image of the sample, etc.

The display unit 140 may include at least one of liquid crystal displays (LCD), thin film transistor-liquid crystal displays (TFT-LCD), organic light-emitting diodes (OLED), flexible displays, and 3D displays.

The user input unit 150 generates an input data which allows the user to control the operation of the OCT. The user input unit 150 may be configured as a keypad, dome switch, touch pad (constant pressure/current), jog wheel, jog switch, etc.

The controller 160 typically controls the general operation of the OCT.

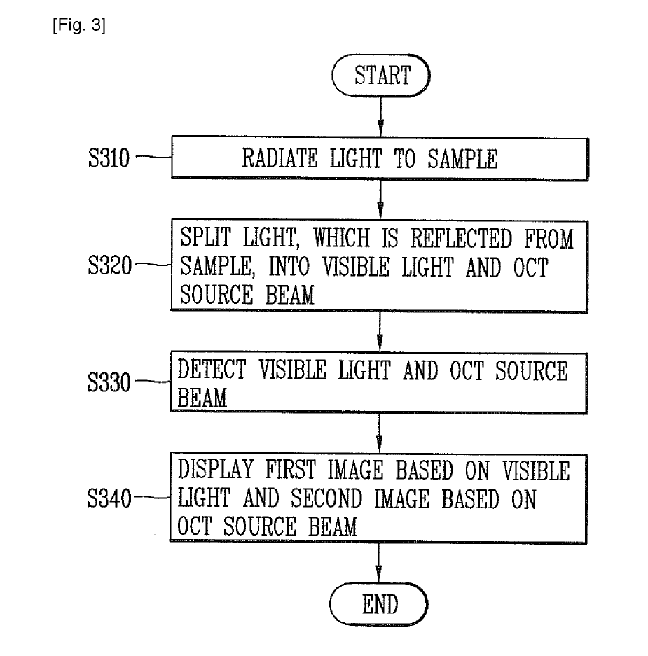

FIG. 3

FIG. 3 is a flowchart showing an example of a method of the OCT displaying an image of a sample.

The OCT can radiate light to the sample (step S310).

The object photographed by the OCT is defined as the sample. For example, the sample can be a human body.

The light radiated to the sample can be reflected.

The OCT can split the light, which is reflected from the sample, into visible light and OCT source beam (step S320).

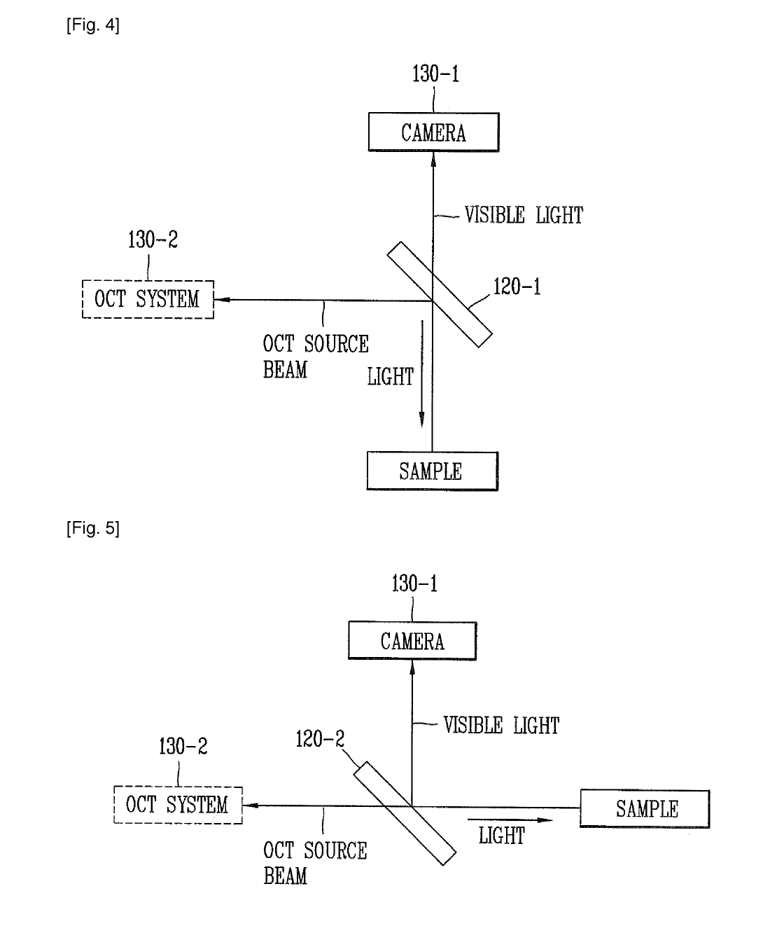

FIGS. 4 and 5

FIGS. 4 and 5 are views showing an example of a method of the OCT splitting light reflected from the sample.

As shown in FIGS. 4 and 5, the OCT can radiate light to the sample. In turn, the radiated light can be reflected from the sample.

The reflected light can be incident on the light splitting unit 120. The light splitting unit may be configured as a dichroic mirror. The dichroic mirror, which is a reflecting mirror consisting of thin material layers with different refractive indices, has a characteristic of reflecting light in specific ranges and transmitting light in another specific ranges. The light splitting unit 120 can transmit the incident light of specific wavelengths and reflect the incident light of another specific wavelengths, using this characteristic. Meanwhile, the light splitting unit may be configured with a dichroic prism, etc. Alternatively, the light splitting unit may be made of various materials which can reflect light of specific wavelengths and transmit light of another specific wavelengths.

Referring to FIG. 4, the light splitting unit 120-1 can reflect the OCT source beam and transmit the visible light. Accordingly, the light splitting unit 120-1 can split the incident light, which is reflected from the sample, into the OCT source beam and the visible light. The OCT may be configured in such a manner that the visible light and the OCT source beam can be incident on the detection unit 130. More particularly, the visible light can be incident on the visible light camera 130-1, while the OCT source beam can be incident on the OCT system 130-2.

Referring to FIG. 5, the light splitting unit 120-2 can reflect the visible light and transmit the OCT source beam. Accordingly, the light splitting unit 120-2 can split the incident light, which is reflected from the sample, into the OCT source beam and the visible light. The OCT may be configured in such a manner that the visible light and the OCT source beam can be incident on the detection unit 130. More particularly, the visible light can be incident on the visible light camera 130-1, while the OCT source beam can be incident on the OCT system 130-2.

In the meantime, the light splitting unit 120 can split only wavelengths of some band among the wavelengths of the visible light band. Not the entire visible light band is needed for the user to check the sample with his/her naked eyes. Therefore, it is possible to split wavelengths of some band which allows the user to check the sample with his/her naked eyes.

In turn, the OCT 100 can detect the visible light and the OCT source beam (step S330).

The detection unit 130 may include a visible light camera 130-1, an OCT system 130-2, etc. Accordingly, the visible light camera 130-1 can acquire a visible image (first image) based on the visible light, while the OCT system 130-2 can acquire a tomographic image (second image) based on the OCT source beam.

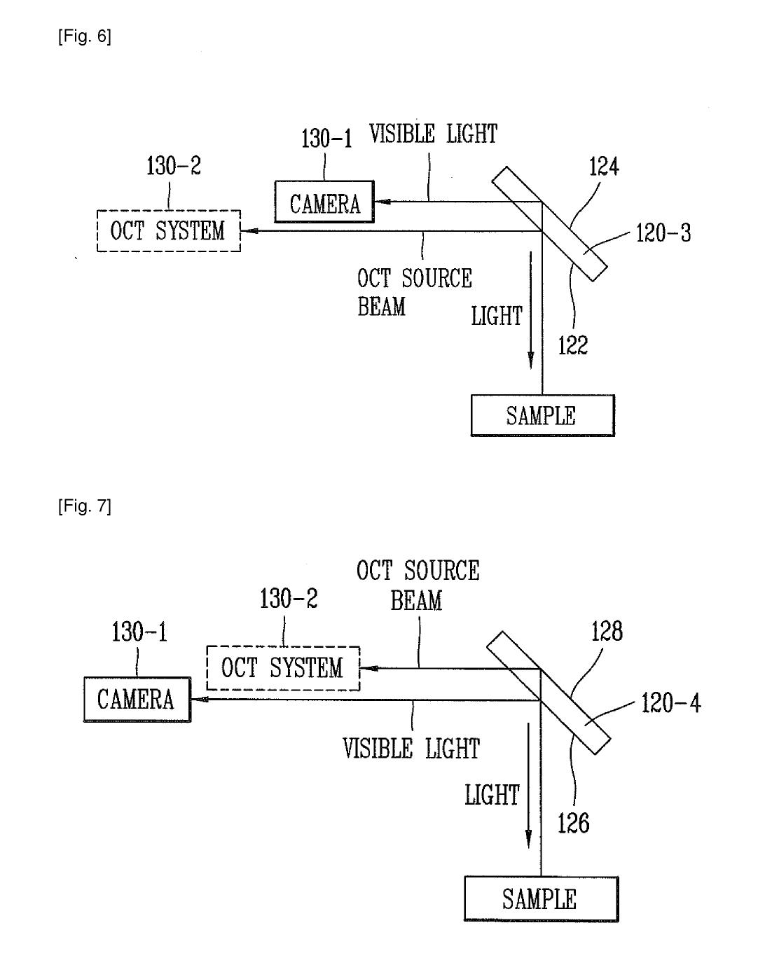

FIGS. 6 and 7

FIGS. 6 and 7 are views showing a further example of the method of the OCT splitting light reflected from the sample.

As shown in FIGS. 6 and 7, the OCT can radiate light to the sample. Then, the radiated light can be reflected from the sample.

The reflected light can be incident on the light splitting unit 120. The light splitting unit 120 may include a panel, wherein one surface of the panel may include an object which reflects and/or transmits certain light, and the other surface of the panel may include an object which reflects and/or transmits another certain light.

Referring to FIG. 6, the light splitting unit 120-3 can reflect the OCT source beam first, and then reflect the visible light. For example, one surface 122 of the light splitting unit 120-3 may include an object which reflects the OCT source beam (e.g., infrared light having a wavelength of 800-1400 nm) and transmits the visible light. In addition, the other surface 124 of the light splitting unit 120-3 may include an object which reflects the visible light.

As a result, the light splitting unit 120-3 can use one surface 122 of the panel to reflect the OCT source beam among the incident light reflected from the sample. In turn, the reflected OCT source beam can be incident on the OCT system 130-2. Alternatively, the light splitting unit 120-3 can use one surface 122 of the panel to transmit the visible light among the incident light reflected from the sample.

The transmitted visible light can be reflected from the other surface 124 of the panel and be incident on the camera 130-1.

In other words, this method allows the light splitting unit 120-3 to split the light, which is reflected from the sample, into the OCT source beam and the visible light.

Referring to FIG. 7, the light splitting unit 120-4 can reflect the visible light first, and then reflect the OCT source beam. For example, one surface 126 of the light splitting unit 120-4 may include an object which reflects the visible light and transmits the OCT source beam (e.g., infrared light having a wavelength of 800-1400 nm). In addition, the other surface 128 of the light splitting unit 120-4 may include an object which reflects the OCT source beam.

As a result, the light splitting unit 120-4 can use one surface 126 of the panel to reflect the visible light among the incident light reflected from the sample. In turn, the reflected visible light can be incident on the camera 130-1. Alternatively, the light splitting unit 120-4 can use one surface 128 of the panel to transmit the OCT source beam among the incident light reflected from the sample.

The transmitted visible light can be reflected from the other surface 128 of the panel and be incident on the OCT system 130-2.

In other words, this method allows the light splitting unit 120-4 to split the light, which is reflected from the sample, into the OCT source beam and the visible light.

In the meantime, the OCT 100 can detect the visible light and the OCT source beam (step S330).

The detection unit 130 may include a visible light camera 130-1, an OCT system 130-2, etc. Accordingly, the visible light camera 130-1 can acquire a visible image (first image) based on the visible light, while the OCT system 130-2 can acquire a tomographic image (second image) based on the OCT source beam.

In turn, the OCT 100 can display the first image and the second image (step S340).

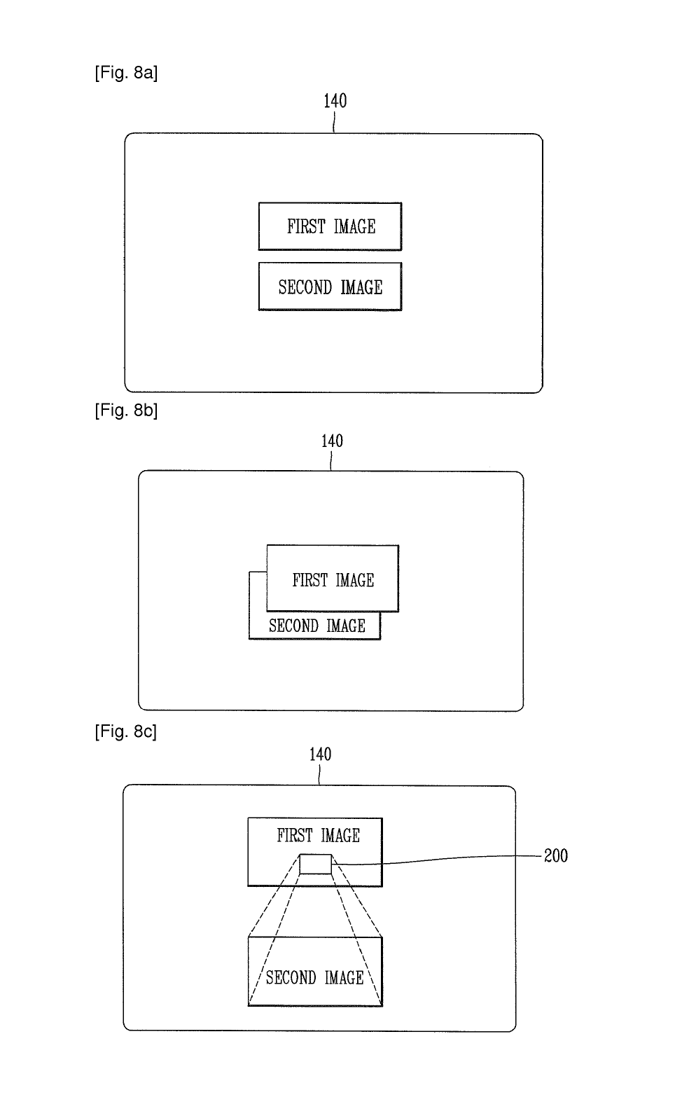

FIG. 8

FIG. 8 is a view showing an example of a method of the OCT displaying the first image and the second image.

As shown in FIG. 8, the display unit 140 can display the visible image (first image) and the tomographic image (second image), which are acquired by the detection unit. Here, one display unit can display both the visible image and the tomographic image of a certain point photographed by the OCT 100. And, one display unit can simultaneously display both the visible image and the tomographic image of a certain point photographed by the OCT 100.

The user can precisely check the photographing portion of the OCT by comparing the first image with the second image. Here, the user can check the real image of the observing point of the sample through the first image. It is thus not necessary for the user to directly check the observing point of the OCT with his/her naked eyes.

Referring to FIG. 8a, the display unit 140 can display the first image, which is a visible image of a certain region of the sample, and the second image, which is a tomographic image of the certain region of the sample, on the top and bottom. That is, the tomographic image of the display region corresponding to the first image can be the second image.

Referring to FIG. 8b, the display unit 140 can display the first image, which is a visible image of a certain region of the sample, and the second image, which is a tomographic image of the certain region of the sample, in the overlapping manner.

Referring to FIG. 8c, the display unit 140 can display the first image, which is a visible image of a certain region of the sample, and the second image, which is a tomographic image of part of the certain region of the sample. That is, the tomographic image of part of the display region corresponding to the first image can be displayed as the second image. In this manner, the user can intuitively check which point of the entire sample region is the point displayed as the tomographic image.

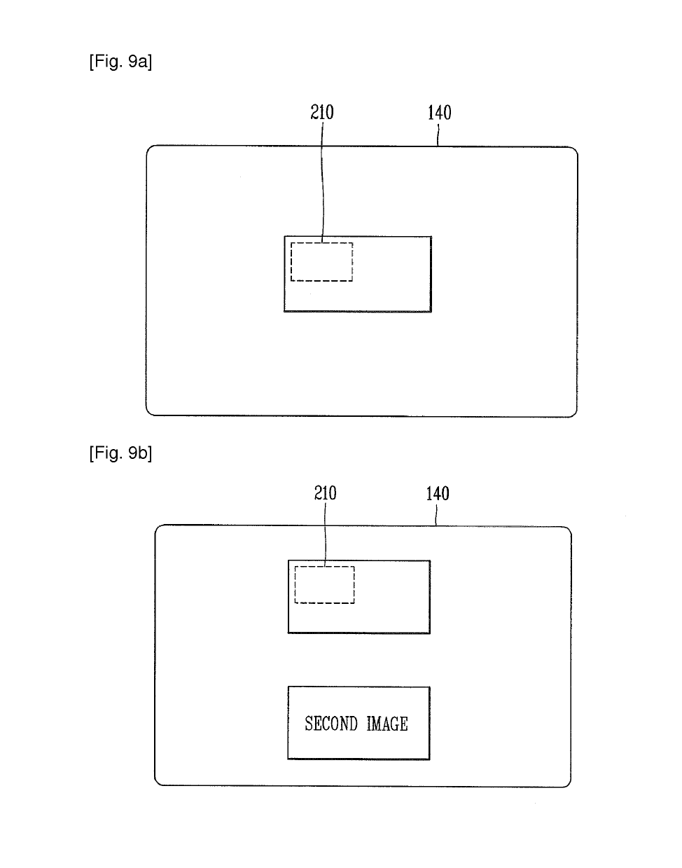

FIG. 9

FIG. 9 is a view showing another example of the method of the OCT displaying the first image and the second image.

In the step of acquiring the first and second images, the detection unit 130 can acquire images corresponding to a wide region.

Therefore, as shown in FIG. 9a, first of all, the display unit 140 can display a visible image (first image) of a wide region, which is acquired by the detection unit.

Here, the user input unit 150 can receive a user s input of selecting some region 210 on the displayed screen. This region 210 may be a region of which the user wants to check a tomographic image.

In this case, the user firstly checks the visible image of the wide region, and then selects a specific region to check its tomographic image, so that the user can precisely select a desired point to check its tomographic image.

Referring to FIG. 9b, the display unit 140 can display only the tomographic image of the selected region 210 according to the user s input.

As such, it is possible for the user to precisely and fully check the observed region by displaying the visible image of the wide region and then displaying the tomographic image of the narrow region according to the users input.

Meanwhile, as the region selected from the first image varies, the second image displayed on the display unit 140 also varies.

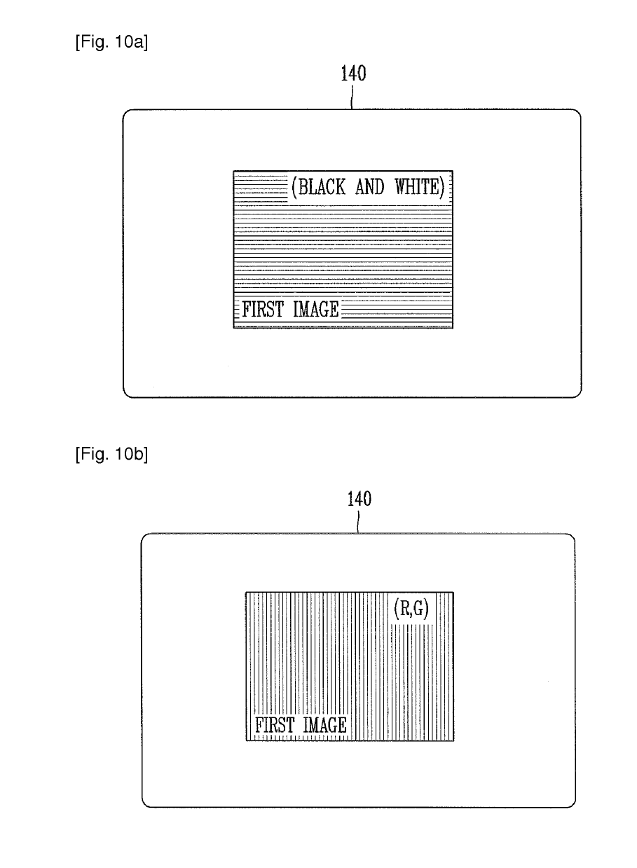

FIG. 10

FIG. 10 is a view showing a further example of the method of the OCT displaying the first image.

Not the entire visible light band is needed for the user to check the sample with his/her naked eyes. Therefore, the light splitting unit 120 can split only wavelengths of some band among the wavelengths of the visible light band. Alternatively, the detection unit 130 can acquire a visible image using only wavelengths of some band among the wavelengths of the visible light band.

In this case, as shown in FIG. 10a, the display unit 140 can display a first image, which is a black and white image.

Alternatively, as shown in FIG. 10b, the display unit 140 can display a first image using only R and G signals.

As set forth herein, if necessary, the OCT can use only wavelengths of some band among the visible light rays to allow the user to check the sample.

The above-described methods according to the embodiments of the present invention may be used individually or in combination. In addition, the steps of one embodiment may be used individually or in combination with the steps of another embodiment.

Moreover, the methods described herein can be implemented in a recording medium, such as a computer or the like, by using, e.g., software, hardware, or a combination thereof.

In hardware implementation, the methods described herein can be implemented by at least one of application specific integrated circuits (ASIC), digital signal processors (DSP), digital signal processing devices (DSPD), programmable logic devices (PLD), field programmable gate arrays (FPGA), processors, controllers, micro-controllers, microprocessors, and other electric units.

In software implementation, the procedures and functions described herein can be implemented by separate software modules. The software modules can be implemented by software codes written in appropriate programming languages. These software codes may be stored in a storage unit and executed by a processor.

1. An optical coherence tomography, comprising:
a light source unit for outputting light;
a light splitting unit for splitting the light, which is reflected from a sample, into visible light and OCT source beam;
a detection unit for detecting the visible light and the OCT source beam; and
a display unit for displaying a first image based on the detected visible light and a second image based on the detected OCT source beam.

2. The optical coherence tomography as claimed in claim 1, wherein the light splitting unit comprises a dichroic mirror.

3. The optical coherence tomography as claimed in claim 2, wherein the dichroic mirror performs a transmission of the visible light and a reflection of the OCT source beam or performs a transmission of the OCT source beam and a reflection of the visible light.

4. The optical coherence tomography as claimed in claim 1, wherein the light splitting unit comprises a panel, one surface of which transmitting the visible light and reflecting the OCT source beam, the other surface of which reflecting the visible light.

5. The optical coherence tomography as claimed in claim 1, wherein the light splitting unit comprises a panel, one surface of which transmitting the OCT source beam and reflecting the visible light, the other surface of which reflecting the OCT source beam.

6. The optical coherence tomography as claimed in claim 1, wherein the detection unit comprises a visible light camera for photographing an image based on the visible light.

7. The optical coherence tomography as claimed in claim 6, wherein the visible light camera comprises at least one of charge coupled device (CCD) cameras and complementary metal-oxide semiconductor (CMOS) cameras.

8. The optical coherence tomography as claimed in claim 1, wherein the detection unit comprises an OCT system for acquiring an image based on the OCT source beam.

9. The optical coherence tomography as claimed in claim 1, wherein the display unit displays the first image and the second image in the overlapping manner.

10. The optical coherence tomography as claimed in claim 1, wherein the first image comprises a visible image of the sample, while the second image comprises a tomographic image of the sample.

11. The optical coherence tomography as claimed in claim 1, further comprising a user input unit for receiving an input of selecting some region of the first image,
wherein the display unit displays the second image corresponding to the selected region.

12. A control method for an optical coherence tomography, comprising:
radiating light to a sample;
splitting the light, which is reflected from the sample, into visible light and OCT source beam;
displaying a visible image of the sample based on the visible light; and
displaying a tomographic image of the sample based on the OCT source beam.

13. The control method as claimed in claim 12, wherein the splitting of light comprises transmitting the visible light and reflecting the OCT source beam at a dichroic mirror.

14. The control method as claimed in claim 12, wherein the splitting of light comprises reflecting the visible light and transmitting the OCT source beam at a dichroic mirror.
